(19) United States
(12) Patent Application Publication
(10) Pub. No.: US 2011/0165196 A1
(43) Pub. Date: Jul. 7, 2011

(54) Method for treatment of crib biting in animals

(76) Inventors: J. Ben Renfroe, Gulf Breeze, FL (US); Daniel M. Carter, Lillian, AL (US)

(21) Appl. No.: 12/655,629

(22) Filed: Jan. 4, 2010

(51) Int. Cl. A61K 39/08

(52) U.S. Cl. 424/239.1

(57) ABSTRACT

The present invention generally relates to methods for treating cribbing behavior in animals. Benefits are obtained by administering to the animals to be treated injections of Botulinum Toxin in the sternocephalic muscle, thereby denervating these muscles and preventing cribbing behavior.

### CROSS-REFERENCES TO RELATED APPLICATIONS

Not Applicable

### STATEMENT REGARDING FEDERALLY SPONSORED RESEARCH OR DEVELOPMENT

Not Applicable

### MICROFICHE APPENDIX

Not Applicable

### BACKGROUND OF THE INVENTION

1. Field of the Invention.

This invention relates to crib biting (cribbing) in animals. More specifically, the invention comprises a method of treating cribbing in animals, particularly horses.

2. Description of the Related Art.

Crib-biting or cribbing is a common oral stereotypy with significant deleterious effects on the health and appearance of horses. In cribbing a horse seizes a fixed object with its incisors and pulls back, flexing the neck, retracting the larynx and drawing air into the cranial esophagus, while usually emitting a characteristic grunt. The vice is common and is thought to be a displacement activity caused by boredom, confinement, isolation or lack of roughage to eat. Although annoyance is the most common complaint by owners, this behavior will damage teeth, result in weight loss, unthriftyness and flatulence. Destruction of confinement hardware is significant.

Economic loss is also significant as cribbing is considered an unsoundness. Approximately 4% of horses admitted to a veterinary referral hospital were for surgical correction of this unsoundness. It has been hypothesized that young horses can learn the behavior but this is unproven. Techniques for control include a cribbing strap to physically prevent the arching of the neck, shock therapy, NMDA receptor antagonists, and surgical intervention.

Surgical techniques have been used trying to correct the crib-biting in horses [bilateral buccostomy (creating bilateral buccal fistulae to prevent development of an oral vacuum), neurectomy of the ventral branch of the spinal accessory nerve (to prevent motor activity of the sternomandibularis muscle), Forssell's procedure (resection of a segment of the omohyoideus, sternothyroideus and stemomandibularis muscles), and the modified Forssell's procedure (transection of a large segment of the ventral branch of the spinal accessory nerve, and transverse and oblique sectioning of the cranial, middle and caudal aspects of the omohyoideus and sternohyoideus muscles).

Another surgical technique that has been used is an neodymium:yttrium-aluminum garnet (Nd:YAG) laser-assisted modified Forssell's surgical technique for treatment of cribbing (crib-biting) in horses. This technique involved an approximately 34-cm ventral median skin incision starting rostral to the larynx and extending caudally. A 10-cm section of the ventral branch of the spinal accessory nerve was removed, using an Nd:YAG laser at 25 W and continuous pulse with a contact, sculpted-fiber tip. After neurectomy, approximately 34-cm sections of the paired omohyoideus and sternothyrohyoideus muscles were removed starting 2 cm rostral to the ventral aspect of the larynx, at the basihyoid bone, using the Nd:YAG laser.

However, these known techniques for treating cribbing suffer from significant defects and are not consistently effective. Shock therapy that is not associated with the behavior only causes the shocked animal pain without changing the behavior. Straps and shock therapy only engage after the animal is well into the behavior sought to be prevented. Moreover, there are significant risks associated with any surgical procedure and associated drug therapies.

Thus, there is a need for a proactive treatment that effectively treats and/or prevents cribbing. This treatment would effectively treat this condition without requiring painful shock therapy, constrictive straps, or invasive surgery with all its associated risks.

### BRIEF SUMMARY OF THE INVENTION

The present invention comprises a method for treatment and/or prevention of cribbing in animals. More specifically, the present invention comprises a method for treatment and/or prevention of cribbing through chemical denervation of the associated muscles via injection of botulinium toxin into these associated muscles.

Clostridium botulinum, the bacteria which produces botulinum toxin, was first identified in the late 1800s. Botulinum toxin consists of seven distinct serotypes, A through G. Of these, Type A (BOTOX, Allergan—Irvine, Calif.; and DYSPORT, Ipsen—Paris, France) and Type B (MYOBLOC, Solstice Neurosciences—South San Francisco, Calif.) are commercially available.

In 1989 Botulinum toxin Type A (BOTOX) was approved for human use by the United States Food and Drug Administration for strabismus (oculomotor dysfunction) and blepharospasm.

Botulinum toxin prevents the release of acetylcholine from the nerve terminal by disrupting the release mechanism of the acetylcholine containing vesicles. This occurs via several mechanisms of action depending on the serotype of botulinum toxin utilized. Blocking the release of acetylcholine from the nerve terminal prevents the contraction of the muscle thereby decreasing the tone of the muscle and the forces exerted at its insertion. When injected into a striated muscle belly the clinical changes are first noted approximately two days later. Peak benefit is in approximately two weeks. The physiologic action dissipates after approximately three months.

The present invention emulates the prior art surgical procedures through the administration of botulinum toxin into the bellies of the sternomandibularis, omohyoideus, and sternohyoideus muscles, thereby providing a non-surgical intervention to prevent cribbing.

### DETAILED DESCRIPTION OF THE INVENTION

The following description contains significant detail regarding the novel aspects of the present invention. It should not be construed, however, as limiting the scope of the invention but rather as providing examples of the preferred method for treatment of the cribbing condition. Thus, the scope of the invention should be fixed by the following claims, rather than by the examples given.

The present invention is directed to the prevention and/or treatment of the cribbing condition in animals. More specifically, the present invention is directed to a method for treating the symptoms of or aiding in the recovery of an afflicted with the condition commonly known as cribbing. Generally, the method of treatment of the invention involves injecting botulinum toxin type A (onabotulinumtoxinA) (BOTOX, Allergan—Irvine, Calif.) to denervate the sternocephalic muscles. This corresponds functionally to the favored surgical technique (modified Forssell's procedure).

The inventor has discovered that administering Botulinum toxin type A is safe and effective for the treatment of cribbing. The disclosed treatment can be used for any animal exhibiting cribbing behavior.

There are seven serotypes of Botulinum Toxin (A through G). The present invention should not be limited to any one serotype of the toxin. In a preferred embodiment, the botulinum toxin (onabotulinumtoxinA) is administered after reconstitution at a desired dilution according to the manufacturer's recommendations. In order to obtain adequate distribution throughout the omohyoideus, sternohyoideus, and sternothyroideus muscles, a dilution of 100 units in 2 cc preservative free saline is preferred. The region to be injected is first prepped utilizing sterile technique. The appropriate muscle is first identified anatomically; the muscle is then accessed utilizing an AMBU NEUROLINE Inoject (Ambu Inc, Glen Burnie Md.) 24 gauge, 3 inch coated needle electrode using electromyography (EMG) guidance. Botulinum Toxin was then injected through this needle in 25 unit aliquots. 400 units of Botulinum Toxin Type A were distributed within the bilateral musculature of each horse as described below.

The reader should note that although the above description and following examples relate to the treatment of cribbing in horses, it is believed that the method for treatment will work as described with any animal.

The present invention may be better understood with reference to the following examples.

### EXAMPLE NO. 1

This horse was a 28 year old gelding quarter horse with a many year history of refractory cribbing. This was partially treated with a “cribbing collar”. Examination showed marked hypertrophy of the sternothyroideus and omohyoideus bilaterally and symmetrically. The horse otherwise showed a normal body habitus and unremarkable examination.

The horse received a total of 400 units of Botulinum toxin type A divided into 14 injections. EMG was utilized and injections were performed utilizing a 24 gauge 3 inch inject able needle electrode. The horse received a total of 100 units in the bilateral sternohyoideus, 150 units in the omohyoideus, and 150 units in the sternothyroideus. The horse tolerated the procedure well with no complications noted.

The horse was evaluated at 13 days after injection. The cribbing collar was removed by the owner with no cribbing reported in the stall. The horse was moved to the paddock where he remained quiet with no cribbing.

At 14 days after injection the horse was again evaluated. He remained without his cribbing collar. He was monitored in his stall for one hour with no cribbing. The horse was observed to walk to his favorite cribbing spot and transiently place his teeth on the board but did not make any movements to begin arching his neck. At 6 weeks post injection, the horse was again evaluated. He remained without his cribbing collar with no cribbing. At 110 days post injection the owner reported onset of a gradual return of stereotypical behavior

### EXAMPLE NO. 2:

This is a 21 year old quarter horse gelding with a many year history of cribbing. This was treated with a cribbing collar. Examination was significant only for hypertrophy of the omohyoideus and sternothyroideus.

The horse received a total of 400 units of Botulinum toxin type A. This was injected utilizing EMG guidance. The horse received 100 units in the bilateral sternohyoideus, 150 units in the omohyoideus, and 150 units in the sternothyroideus. He tolerated the procedure well with no complications noted.

Thirteen days after the initial injection the horse was evaluated. After the cribbing collar was removed the horse was noted to place his teeth on a board and begin to arch his neck. He then abruptly stopped and walked away. Follow-up by phone noted a persistence of the benefit for approximately 3 months before a gradual return of the stereotypy was noted by the owner.

What is claimed is:

1. A method for treating cribbing in an animal comprising:
injecting a Botulinum Toxin, in a therapeutically effective amount sufficient to treat said cribbing condition, into a sternocephalic muscle, said therapeutically effective amount insufficient to cause death or paralysis of said animal when injected as such.

2. A method as defined in claim 1, wherein said Botulinum Toxin is Botulinum Toxin Type A.

3. A method as defined in claim 1, wherein said sternocephalic muscle includes at least one of a omohyoideus muscle, a sternohyoideus muscle, or a sternothyroideus muscle.

4. A method as defined in claim 1, wherein said Botulinum Toxin is administered to said animal in an amount from about 200 Units to 400 Units.

5. A method as defined in claim 4, wherein said Botulinum Toxin is injected into said sternocephalic muscle in 8 to 20 injections.

6. A method as defined in claim 1, wherein said animal is a horse.

7. A method as defined in claim 1, wherein said animal is selected from the group consisting of horses, cattle, camels, goats, pigs, and sheep.

8. A method for preventing cribbing in animals comprising:
injecting a Botulinum Toxin into a sternocephalic muscle in a therapeutically effective amount sufficient to denervate said sternocephalic muscle, said therapeutically effective amount insufficient to cause death or paralysis of said animal when injected as such.

9. A method as defined in claim 8, wherein said Botulinum Toxin is Botulinum Toxin Type A.

10. A method as defined in claim 8, wherein said Botulinum Toxin is administered to said animal in an amount from about 200 Units to 400 Units.

11. A method as defined in claim 12, wherein said sternocephalic muscle includes at least one of an omohyoideus muscle, a sternohyoideus muscle, or a sternothyroideus muscle.

12. A method as defined in claim 10, wherein said Botulinum Toxin is injected into said sternocephalic muscle in four to ten injections.

13. A method for treating a cribbing condition in an animal comprising: administering a plurality of injections of Botulinum Toxin Type A, each of said injections containing a pharmaceutically effective amount of Botulinum Toxin Type A, into a sternocephalic muscle of said animal.
